(19) United States
(12) Patent Application Publication
(10) Pub. No.: US 2015/0376457 A1
(43) Pub. Date: Dec. 31, 2015

(54) METHOD FOR COATING SURFACES

(71) Applicant: NATVI SAS, Corbeil-Essonnes (FR)

(72) Inventor: Robert MICHELOT, Antony (FR)

(73) Assignee: NATVI SAS, Corbeil-Essonnes (FR)

(21) Appl. No.: 14/764,031

(22) PCT Filed: Jan. 31, 2014
(86) PCT No.: PCT/FR2014/050175

§ 371 Date: Jul. 28, 2015

(30) Foreign Application Priority Data
Feb. 1, 2013 (FR) 1350902

(51) Int. Cl. C09D 199/00; B05D 7/00; A61L 31/10

(52) U.S. Cl. CPC C09D 199/00; A61L 31/10; B05D 7/52; A61L 2420/08; A61L 2420/02

(57) ABSTRACT

The invention relates to a method for coating a surface, comprising: a step of bringing the surface into contact with a first composition comprising an amphiphilic compound; and a step of bringing same into contact with a second composition comprising a hydrophilic polymer.

The present invention relates to the technical field of processes for covering surfaces. Functionalization of the surface of materials used for various applications, especially in the medical field, is a determining factor for the interaction of the materials with the environment. However, such a modification is not always simple.

The coating of materials for medical use, which are generally more or less hydrophobic, is often difficult when an aqueous solution comprising a biocompatible product needs to be deposited on the surface thereof. Specifically, due to their hydrophobicity, the wetting of the surface of these materials is imperfect and the aqueous solution has a tendency to adhere nonuniformly and to assemble as droplets.

In order to limit these phenomena, various surface treatments are generally used, so as to cover the hydrophobic surfaces with hydrophilic substances or compounds, allowing good wetting of the surface, and limiting the assembly in the form of droplets.

To date, several processes for covering hydrophobic surfaces are used.

A first type of process consists in activating the surface to be covered and covalently grafting biocompatible hydrophilic molecules thereon.

A pretreatment with a reagent that is capable of modifying the surface may lead to the formation of derivable groups. For example, the hydrolysis of polyesters or polyamides with a strong base or a strong acid leads to alcohol, acid or amine groups [Chollet, C. et al. 2007 Biomolecular Engineering 24, 477-482].

In the case of a chemically inert material, the surface may be activated via more drastic methods using, for example, a plasma under vacuum or at ambient pressure or via ionizing radiation, or alternatively in solution, via the action of free-radical-generating reagents. The transient ions or free radicals can then attach biocompatible molecules, via covalent or ionic bonds [Goddard, J. M; & Hotchkiss J. H. 2007 Progress in Polymer Science 32, 698-625].

A second type of process, illustrated especially in patent application US 2010/0076546, consists in covering the hydrophobic surface with a solution of monomer of a polymerizable hydrophilic molecule. Activation of polymerization is performed by UV irradiation, use of a catalyst or raising of the temperature.

Such processes are, however, difficult to perform and are relatively expensive. Moreover, the use of catalysts, plasma or UV or ionizing rays may impair the surface of the materials, which, in the context of devices for medical use, may lead to degradation or malfunction of the device.

Thus, there is a need for a simple and inexpensive method, which does not impair the properties of the surface to be covered with a biocompatible hydrophilic compound.

A third type of process has been performed, and exploits the self-assembly capacities of certain compounds in solution or at the liquid-solid or liquid-gas interface. However, in the context of processes of this type, the molecules that are capable of self-assembling react via their polar part with the surface to be covered, thus exposing their hydrophobic part. When the polar end of these molecules is linked to the substrate, which is generally obtained by ionic or covalent bonding, the interactions between these molecules reorganize the surface edifice mainly via forces of weak intensity. These lowenergy interactions are capable of forming fibrils organized in rows at the surface of the substrate. The solidity of this monolayer thus arises both from covalent or ionic interactions with the material and from interactions between the molecules themselves.

However, although such a process is simpler than those mentioned above, it has the consequence of increasing the hydrophobicity of the covered surface due to the exposure of the hydrophobic parts of the self-assembled molecules.

Thus, the abovementioned needs remain.

One object of the present invention is to overcome the abovementioned drawbacks. Furthermore, an object of the invention is to provide a simple process for covering a hydrophobic surface with a biocompatible hydrophilic substance.

One of the aims of the invention is the surface treatment of medical devices for the purposes of making them biocompatible, lubricated and, optionally, capable of releasing chemical or biological factors that promote their implantation (antibiotics, growth molecules, cell recognition factors, etc.).

Another object of the invention is directed toward providing covered surfaces that have improved wetting.

Yet another object of the invention has the aim of providing kits for performing the abovementioned process.

The invention relates to a process for covering a hydrophobic surface with a hydrophilic composition, said process comprising:
a. a step of placing in contact of the hydrophobic surface for the purpose of covering it with a first composition comprising
   a solvent, and
   a solute consisting of an amphiphilic compound that is capable of self-assembling and of interacting with said surface, the self-assembly of said compound and the interaction with the surface taking place by means of bonds other than covalent or ionic bonds,
      said solvent being compatible with said compound and said hydrophobic surface,
b. a subsequent step of rinsing with an aqueous solution of said hydrophobic surface covered in the preceding step, and
c. a step of placing in contact of the rinsed surface with a second hydrophilic composition comprising a hydrophilic polymer.

The invention is based on the surprising observation, made by the inventors, that self-assembling amphiphilic compounds are capable, especially in solution in a suitable solvent, of forming oriented layers of molecules, capable of interacting with an inert support without, however, forming covalent or ionic bonds with the substrate. Thus, it is no longer necessary to activate the substrate, which limits its degradation.

The compound used in the context of the invention is an amphiphilic compound, i.e. a compound bearing both at least one hydrophilic group and at least one hydrophobic group.

Molecular self-assembly is the phenomenon via which compounds form by themselves structures with a high degree of organization, without external intervention. It may be considered schematically that these compounds have sufficient affinity to be able to assemble together.

Two types of self-assembly are distinguished, intramolecular and intermolecular. Intramolecular self-assembly often produces complex polymers which have the possibility of adopting a stable and well-defined structural conformation. Intermolecular self-assembly defines the capacity that certain molecules have to form supramolecular assemblies. An example of this type of self-assembly concerns the formation of micelles from surfactants in solution. Self-assembly is generally due to bonds of van der Waals type. The stability of the self-assembly results mainly from thermodynamic factors which favor the ordered forms of matter (neguentropy) relative to the disordered states.

Self-assembly may take place spontaneously, for example in cells (in which the membrane is formed from a self-assembled lipid bilayer) and other biological systems, just as in artificial systems. It generally results in an increase in the internal organization of the system. Self-assembled biological systems, including self-assembled synthetic peptides and other biomaterials, show greater ease of manipulation, of biocompatibility and of functionality. These advantages are directly due to self-assembly starting from biocompatible precursors which create biomaterials synthesized at the nanometric scale. In the first step of the process, only the intermolecular interactions are involved. The forces generated by the self-assembly of these compounds are sufficient to allow the compound to interact with the substrate, and to remain “attached” thereto, without it being necessary to involve physicochemical interactions therewith.

In other words, there is no covalent bonding between the self-assembling compound and the surface covered therewith. Similarly, there are no ionic bonds between the self-assembling compound and the surface covered therewith.

In the first step of the process according to the invention, a solution of solvent comprising a self-assembling compound is placed in contact with the surface to be covered. “Said solvent is compatible with said compound and said hydrophobic surface”, which means that the self-assembling compound is soluble in said solvent, and that the solvent does not exert any effect drastically impairing the nature and surface of the substrate to be covered.

In an advantageous embodiment, the solvent used is capable of exerting gentle abrasion of the surface to be covered, such that microdepressions may be created at the surface of said surface.

Advantageously, the surface is precleaned and degreased so as to remove traces of compounds that may impair the self-assembly of the compounds.

Depending on the nature of the surface, it may be cleaned and degreased with a suitable solvent. A person skilled in the art is capable of determining the solvent or mixtures of solvents required. The experimental section below gives certain indications.

In the invention, the term “solvent that is compatible with said compound” means a solvent that is capable of dissolving said self-assembling amphiphilic compound. A person skilled in the art can readily determine the solvent, or the mixture of solvents, that are the most appropriate for dissolving the amphiphilic compound. The term “solvent that is compatible with said hydrophobic surface” means a solvent that is capable of not degrading or impairing the hydrophobic surface.

In one embodiment of the invention, the first step of the process may be performed directly during the manufacture of the hydrophobic surface to be covered. For example, the self-assembling compound, considered as an additive in the form of powder or granules, optionally in the form of a solution, especially of a highly concentrated solution, is added to granules of hydrophobic polymers, and the mixture thus obtained is then subjected to melting and then to extrusion. In this embodiment, during cooling, the self-assembling compounds migrate slowly toward the superficial parts of the surface undergoing formation, and the polar parts of the compound are exposed at the exterior of the surface.

In yet another advantageous embodiment, the surface to be covered is treated with a solvent for the polymer, for a given time, so that microabrasion appears on the superficial parts of the surface. Such abrasions allow the diffusion and regrouping of the self-assembling compound and the exposure of its hydrophilic part toward the exterior of the substrate.

In the second phase, a biocompatible compound or polymer in the form of a dilute gel or a very dispersed polymer coats the substrate already covered with self-assembling molecules.

The hydrophobic surfaces to be covered via the process of the invention are generally made of polymers of the thermoplastic type such as polyethylene (PE), polypropylene (PP), polyethylene terephthalate (PET), polybutylene terephthalate (PBT), polyoxymethylene (POM), polyarylate (PAr), polyether ketone (PEK), fluoropolymers (for example: polyvinylidene fluoride (PVDF), cycloolefinic polymers (COC) (for example the polymers sold under the brand name TOPAS), cycloolefinic copolymers (COP) (for example the copolymers sold under the brand name Zeonex), polytetrafluoroethylene (PTFE), polychlorotrifluoroethylene (PCTFE)), polysulfone (PSU), ethylene-propylenediene monomer (EPDM), or of the thermosetting type such as synthetic rubbers (for example: halobutyl rubbers, nitrile rubbers, polyisoprenes and polychloroprenes) or alternatively of the thermoplastic elastomer type (for example: the EPDM-PP copolymer sold under the brand name Santoprene, styrene-ethylene-butylene-styrene (SEBS) block copolymer, etc.).

Other surfaces such as stainless steel, gold, titanium, platinum, aluminum, nickel and titanium alloy or nitinol, tantalum or silicones may be covered according to the process of the invention. The abovementioned list is not limiting, and a person skilled in the art understands that it is a matter of hydrophobic surfaces that may be used especially in the field of devices for medical use.

In a second step of the process according to the invention, the surface covered with the abovementioned amphiphilic compound is washed with an aqueous solution, especially water, advantageously distilled water, in particular sterile water, or a mixture of solvents with water.

Such a washing step is essential because it improves the organization of the self-assembling molecules at the surface of the substrate.

Once washed, the surface may advantageously be dried.

In the third step of the process according to the invention, the hydrophobic surface, covered with the self-assembling amphiphilic compound that has been washed and optionally dried, is covered, or coated, with a second composition comprising at least one, or more, hydrophilic polymers.

The second composition comprising at least one, or more, hydrophilic polymers is conventionally dissolved, and placed in contact with the surface washed and optionally dried beforehand in the preceding step.

The second compositions comprising at least one hydrophilic polymer which are advantageous according to the invention comprise, without this being limiting, anionic or cationic polymers, acidic polymers, amine polymers or amino acids, and biologically and/or pharmaceutically acceptable salts thereof.

It is also possible to have available mixtures of the abovementioned polymers. Examples that may be mentioned include: collagen, oxidation-modified collagen, polysaccharides, alginates, hyaluronic acid, polylysine, etc. Such examples are given as a guide and should not be considered as limiting the scope of the invention.

It is advantageous in the invention to choose the hydrophilic polymer such that it has affinity for the amphiphilic self-assembling compound.

Thus, it will be advantageous to use polycations, for example polyamines, histones (proteins surrounding the DNA of eukaryotic cells and rich in basic amino acids), chitosan, polylysine, etc. when the self-assembling compound comprises an anionic polar part and to choose polyanions, such as hyaluronic acid, when the self-assembling compound comprises a cationic polar part.

Advantageously, the hydrophilic polymer interacts with the self-assembling amphiphilic compound by means of hydrogen bonds.

To increase the solidity of covering of the surface, and advantageously to control the biodegradation, it is possible to perform crosslinking of said hydrophilic polymer, with suitable reagents. A person skilled in the art, as a function of the polymer under consideration, is capable of determining the appropriate crosslinking methods.

In one advantageous embodiment, the invention relates to a process according to the preceding definition, in which the amphiphilic compound is of formula I below:

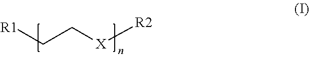

in which
n is other than zero,
X is a functional group chosen from an amino group —NR3-, an amido group, an imino group, a carbonyl group, a carboxyl group, an ester group, a linear aromatic or polyaromatic group, a sulfonate group, a sulfate group, a phosphate group or a phosphonate group,
in which R3 is a hydrogen, an amine or a polyethyleneimine,
R2 is a hydrogen atom or a methyl, ethyl or propyl group or equal to R1,
and R1 is such that
   if n=1, R1 is a saturated or unsaturated C10-C24, especially C12-C20, preferentially C14-C18, and in particular C16 alkyl, and
   if n>1, R1 is a hydrogen atom or a saturated or unsaturated, C10-C24, especially C12-C20, preferentially C14-C18, in particular C16 alkyl, or n preferentially ranging from 8 to 650.

Self-assembly of the amphiphilic compound is ensured by the aliphatic or aromatic part thereof. Thus, the more the compound comprises a hydrophobic part, the greater its capacity to self-assemble, to a certain extent.

When n=1 and R2 is a hydrogen, R1 is a saturated or unsaturated C10-C24 alkyl, which means that R1 is a C6, C7, C8, C9, C10, C11, C12, C13, C14, C15, C16, C17, C18, C19, C20, C21, C22, C23 or C24 linear or branched alkyl.

The advantageous compounds are chosen from the following:
when X═COO—, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid,
when X═NH—, tridecanamine, tetradecanamine, pentadecanamine, hexadecanamine, heptadecanamine, octadecanamine, nonadecanamine, eicosanamine, heneicosanamine, docosanamine, tricosanamine, tetracosanamine, pentacosanamine and hexacosanamine, in particular octadecanamine or sterarylamine is an advantageous self-assembling compound.

Advantageously, the invention relates to a process for covering a hydrophobic surface with a hydrophilic composition, said process comprising:
a. a step of placing in contact of the hydrophobic surface for the purpose of covering it with a first composition comprising
   a solvent, and
   a solute consisting of an amphiphilic compound of formula I below:

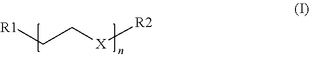

in which
n is equal to 1,
X is a functional group chosen from an amino group, an amido group, a carbonyl group, a carboxyl group, an ester group, a linear aromatic or polyaromatic group, a sulfonate group, a sulfate group, a phosphate group or a phosphonate group,
R2 is a hydrogen atom or a methyl, ethyl or propyl group or equal to R1,
and R1 is a saturated or unsaturated, C10-C24, especially C12-C20, preferentially C14-C18, in particular C16 alkyl, said solvent being compatible with said compound and said hydrophobic surface,
b. a subsequent step of rinsing with an aqueous solution of said hydrophobic surface covered in the preceding step, and
c. a step of placing in contact of the rinsed surface with a second hydrophilic composition comprising a hydrophilic polymer.

In another advantageous embodiment, the invention relates to the abovementioned process, in which the amphiphilic compound chosen from the compounds having the following formula:

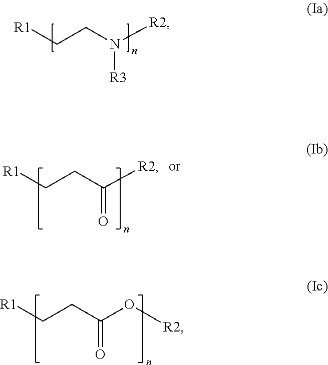

in which R1 and R2 are as defined previously, and in which R3 is a hydrogen atom, an amine, an imine, an acid, an alcohol, a polymer, especially polyethyleneimine.

In another embodiment, the invention relates to a process according to the abovementioned definition, in which said amphiphilic compound is chosen from the compounds having the following formulae:

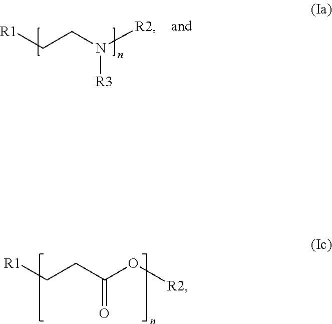

in which R2 and R3 are hydrogen atoms, and R1 is a saturated or unsaturated C10-C24, especially C12-C20, preferentially C14-C18, in particular C16 alkyl.

In yet another advantageous embodiment, the invention relates to the abovementioned process, in which the amphiphilic compound is of formula 1a, such that
if n=1, R2 and R3 are hydrogen atoms, and R1 is a linear or branched, in particular linear, 010-C24 alkyl, and
if n>1, R1, R2 and R3 are hydrogen atoms or polyethyleneimines.

When n>1, and especially from 8 to 650, advantageous compounds of the invention are linear or branched polyethyleneimines, optionally dendrimers. Examples of polyethyleneimines used in the context of the invention are presented below:

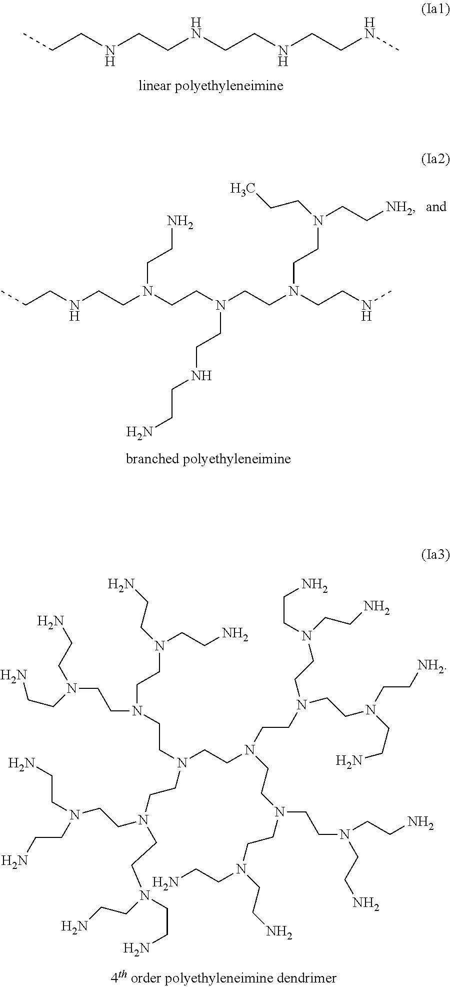

The polyethyleneimines used in the context of the invention may also be modified so as to reduce their relative toxicity. In particular, polyethyleneimine conjugated with polyethylene glycol may be advantageously used in the context of the process according to the invention.

In an advantageous embodiment, the invention relates to the process as defined above, in which said compound is of formula Ia.

In another advantageous embodiment, the invention relates to the process as defined above, in which said compound is chosen from stearylamine or stearic acid, or a salt thereof, or in which said compound is branched polyethyleneimine, advantageously of low molecular weight, especially from about 10 to about 100 kDa, preferentially from about 20 to about 80 KDa, in particular from about 25 kDa to 40 kDa, in particular 25 kDa.

In an advantageous embodiment, the invention relates to a process for covering a hydrophobic surface with a hydrophilic composition, said process comprising:
a. a step of placing in contact of the hydrophobic surface for the purpose of covering it with a first composition comprising
   a solvent, and
   a solute consisting of an amphiphilic compound that is capable of self-assembling and of interacting with said surface, the self-assembly of said compound and the interaction with the surface taking place by means of bonds other than covalent or ionic bonds, said compound being chosen from stearylamine or polyethyleneimine, especially of low molecular weight, said solvent being compatible with said compound and said hydrophobic surface,
b. a subsequent step of rinsing with an aqueous solution of said hydrophobic surface covered in the preceding step, and
c. a step of placing in contact of the rinsed surface with a second hydrophilic composition comprising a hydrophilic polymer.

Another advantageous embodiment of the invention relates to a process as defined previously, in which said amphiphilic compound is chosen from stearylamine or stearic acid, or a salt or a derivative thereof.

Stearylamine or stearic acid are advantageous since they are not toxic to human or animal health. In contrast with other self-assembling amphiphilic compounds of the invention, the compounds of formula I(a) or I(c) in which R2 and R3 are hydrogen atoms, and R1 is a saturated or unsaturated C10-C24, especially C12-C20, preferentially C14-C18, in particular C16 alkyl, and thus stearylamine or stearic acid, may be ingested or applied to the human or animal body without danger.

It is also advantageous to use stearic acid salts that do not have any toxicity either. The ions associated with the salts of the invention are especially alkali metal ions such as lithium ions (Li⁺), sodium ions (Na⁺), potassium ions (K⁺), alkaline-earth metal ions such as calcium ions (Ca²⁺), magnesium ions (Mg²⁺), iron 11 ions (Fe²⁺), zinc ions (Zn²⁺) and trivalent ions such as aluminum ions (Al³⁺) and chromium III ions (Cr³⁺).

Stearic acid may also be combined with cationic molecules such as ammonium ions, especially quaternary ammoniums, or protonated primary or secondary amines, this list not being limiting.

Advantageously, the invention relates to a process as defined above, in which said hydrophilic polymer is chosen from hyaluronic acid, an alginate, oxidized cellulose, chitosan or oxidized starch or a derivative thereof. These polymers are given as examples, and this list should not be considered as limiting.

Hyaluronic acid or derivatives thereof are particularly advantageous in the context of devices for medical use.

The abovementioned hyaluronic acid derivatives may be, for example, without, however, limiting the scope of the invention, oxidized hyaluronic acid obtained by treatment of hyaluronic acid with sodium periodate, as mentioned in Kristiansen K. A. et al. [Carbohydrate Research 2010, 345, 1264-1271], or hyaluronic acid modified with adipic dihydrazide in the presence of a coupling agent, as described in Prestwich G. D. et al. [Journal of Controlled Release 1998, 53, 93-103]. A person skilled in the art may also refer to the following documents for determining the appropriate modifications of hyaluronic acid: Bullpitt P and Aeschlimann D, J. Biomed Mater Res. 1999, 47, 152-169 and Schanté C E et al. Carbohydrate Polymers 2011, 85, 469-489.

When the amphiphilic compound is stearic acid, and in the case of covering with hyaluronic acid, it is advantageous to use multivalent cations (for example chromium III ions, which are not toxic, or aluminum ions) which act as crosslinking agents. Advantageously, these cations are applied to the surface covered with the amphiphilic compound before coating with the hydrophilic polymer.

Advantageously, the invention relates to the process as defined above, said hydrophilic polymer is hyaluronic acid at a concentration of from 0.1% to 0.5% or from 0.1% to 2% in water.

In an even more advantageous embodiment, the invention relates to a process for covering a hydrophobic surface with a hydrophilic composition as claimed in any one of the preceding claims, said process comprising
a. a step of placing in contact of the hydrophobic surface with a first composition comprising, or consisting essentially of,
   a suitable solvent, especially heptane, dichloromethane, 2-methyltetrahydrofuran, dimethylformamide or dimethylacetamide, or a mixture thereof, especially dimethylformamide mixed with dichloromethane, and
   stearylamine, especially at a concentration of from 0.1% to 0.5% or from 0.1% to 3%, preferably at 0.3%, in said solvent,

said solvent being compatible with said compound and said hydrophobic surface,
b. a step of rinsing with water of said hydrophobic surface covered in the preceding step, and
c. a step of placing in contact of the surface obtained in the preceding step with a second hyaluronic acid composition at a concentration of from 0.1% to 0.5% or from 0.1% to 2% in water.

In an even more advantageous embodiment, the invention relates to a process for covering a hydrophobic surface with a hydrophilic composition as defined above, said process comprising
a. a step of placing in contact of the hydrophobic surface with a first composition comprising, or consisting essentially of,
   a suitable solvent, especially heptane, dichloromethane, 2-methyltetrahydrofuran, dimethylformamide or dimethylacetamide, or a mixture thereof, especially dimethylformamide mixed with dichloromethane, and
   stearic acid at a concentration of from 0.1% to 3%, preferably at 0.3%, in said solvent,

said solvent being compatible with said compound and said hydrophobic surface,
b. a step of rinsing with water of said hydrophobic surface covered in the preceding step, and
c. a step of placing in contact of the surface obtained in the preceding step with a second hyaluronic acid composition at a concentration of from 0.1% to 0.5% or from 0.1% to 2% in water.

In another even more advantageous embodiment, the invention relates to a process for covering a hydrophobic surface with a hydrophilic composition as defined above, said process comprising
a. a step of placing in contact of the hydrophobic surface with a first composition comprising, or consisting essentially of,
   a suitable solvent, especially heptane, dichloromethane, 2-methyltetrahydrofuran, dimethylformamide or dimethylacetamide, or a mixture thereof, especially dimethylformamide mixed with dichloromethane, and
   stearic acid or stearylamine at a concentration of from 0.1% to 3%, preferably at 0.3%, in said solvent,

said solvent being compatible with said compound and said hydrophobic surface,
b. a step of rinsing with water of said hydrophobic surface covered in the preceding step, and
c. a step of placing in contact of the surface obtained in the preceding step with a second composition comprising oxidation-modified hyaluronic acid, or hyaluronic acid modified with adipyl dihydrazide, or oxidized cellulose, or a mixture thereof at a concentration of from 0.1% to 0.5% or from 0.1% to 2% in water.

In the invention, “oxidation-modified hyaluronic acid, hyaluronic acid modified with adipyl dihydrazide, or oxidized cellulose, or a mixture thereof” means that the following compositions may be envisaged:
oxidation-modified hyaluronic acid,
hyaluronic acid modified with adipyl dihydrazide,
oxidized cellulose,
oxidation-modified hyaluronic acid and hyaluronic acid modified with adipyl dihydrazide,
oxidation-modified hyaluronic acid and oxidized cellulose,
hyaluronic acid modified with adipyl dihydrazide, and oxidized cellulose, and
oxidation-modified hyaluronic acid and hyaluronic acid modified with adipyl dihydrazide and oxidized cellulose.

Another embodiment, the invention relates to a process for covering a hydrophobic surface with a hydrophilic composition as claimed in any one of the preceding claims, said process comprising
a. a step of placing in contact of the hydrophobic surface with a first composition comprising, or consisting essentially of,
   a suitable solvent, especially water, dichloromethane, 2-methyltetrahydrofuran, dimethylformamide, dimethylacetamide or N,n′-dimethylpropyleneurea (DMPU), or alternatively a mixture thereof, in particular dichloromethane or 2-methyltetrahydrofuran as a mixture with dimethylformamide or dimethylacetamide or N,n′-dimethylpropyleneurea (DMPU), and
   stearic acid, especially at a concentration of from 0.3% to 1%, in said solvent,

said solvent being compatible with said compound and said hydrophobic surface,
b. a step of rinsing with water of said hydrophobic surface covered in the preceding step, and
c. a step of placing in contact of the surface obtained in the preceding step with a second composition comprising hyaluronic acid at a concentration of from 0.1% to 0.5% in water and chromium III ions, especially basic chromium III acetate, in particular at 0.5% in water.

In another even more advantageous embodiment, the invention relates to a process for covering a hydrophobic surface with a hydrophilic composition as claimed in any one of the preceding claims, said process comprising
d. a step of placing in contact of the hydrophobic surface with a first composition comprising, or consisting essentially of,
   a suitable solvent, especially water, dichloromethane, 2-methyltetrahydrofuran, dimethylformamide, dimethylacetamide or N,n′-dimethylpropyleneurea (DMPU), or alternatively a mixture thereof, in particular dichloromethane or 2-methyltetrahydrofuran as a mixture with dimethylformamide or dimethylacetamide or N,n′-dimethylpropyleneurea (DMPU)
   polyethyleneimine, especially at a concentration of from 0.3% to 1%, preferably at 0.5%, in said solvent,

said solvent being compatible with said compound and said hydrophobic surface,
e. a step of rinsing with water of said hydrophobic surface covered in the preceding step, and
f. a step of placing in contact of the surface obtained in the preceding step with a second hyaluronic acid composition at a concentration of from 0.1% to 0.5% in water.

In the advantageous embodiment above, step a. may be performed by incorporating stearylamine at a concentration of from 0.1% to 3%, or stearic acid at a concentration of from 0.1% to 3%, in a material made of polypropylene before molding. On cooling, the stearylamine or the stearic acid migrates slowly to the surface and then combines to form the first layer.

The invention also relates to a hydrophobic surface covered with hyaluronic acid, which may be obtained according to the process as defined above.

The surface covered as defined above comprises one or more layers of amphiphilic compound as defined above, in a determined thickness, which are themselves covered with one or more layers of hydrophilic polymer(s).

The invention also relates to a hydrophobic surface covered with a first layer of an amphiphilic compound that is capable of self-assembling, especially stearylamine, stearic acid or polyethyleneimine, the self-assembly of said compound and the interaction with the surface taking place by means of bonds other than covalent or ionic bonds, said first layer being covered with a second layer comprising or essentially consisting of a hydrophilic polymer, especially hyaluronic acid, which is advantageously crosslinked.

The invention also relates to the use of a complex of stearylamine and of hyaluronic acid for covering hydrophobic surfaces, the compounds forming said complex being linked via bonds other than covalent bonds.

The invention also relates to the use of a complex of polyethyleneimine and of hyaluronic acid for covering hydrophobic surfaces, the compounds forming said complex being linked via bonds other than covalent bonds.

Advantageously, the invention relates to a hydrophobic surface covered with a first layer of an amphiphilic compound of formula I below:

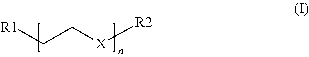

in which
n is equal to 1,
X is a functional group chosen from an amino group, an amido group, a carbonyl group, a carboxyl group, an ester group, a linear aromatic or polyaromatic group, a sulfonate group, a sulfate group, a phosphate group or a phosphonate group,
R2 is a hydrogen atom or a methyl, ethyl or propyl group or is equal to R1,
and R1 is a saturated or unsaturated C10-C24, especially C12-C20, preferentially C14-C18, in particular C16 alkyl,
said first layer being covered with a second layer comprising or consisting essentially of a hydrophilic polymer.

In the invention, said hydrophilic polymer is as defined previously and hereinbelow and possibly includes the presence of ions, especially chromium III ions.

In another advantageous embodiment, the invention relates to a hydrophobic surface as defined previously, in which said amphiphilic compound is chosen from the compounds having the following formulae:

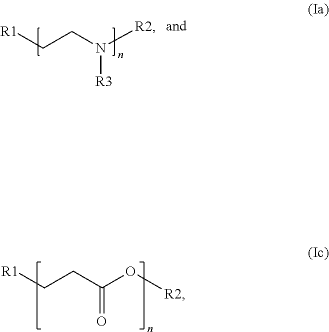

in which R2 and R3 are hydrogen atoms, and R1 is a saturated or unsaturated C10-C24, especially C12-C20, preferentially C14-C18, in particular C16 alkyl.

In yet another embodiment, the invention relates to a hydrophobic surface as defined previously, in which said amphiphilic compound is chosen from stearylamine or stearic acid, or a salt or a derivative thereof.

Even more advantageously, stearic acid is used at a concentration of from 0.1% to 3%, preferably at 0.3%, whereas stearylamine is used at a concentration of from 0.1% to 3%, preferably 0.3%.

The invention moreover relates to the use of a composition essentially comprising stearylamine or stearic acid for covering hydrophobic surfaces with hyaluronic acid, alginate, oxidized cellulose, oxidized starch or a derivative thereof or a mixture thereof.

The invention moreover relates to the use of a set of compositions for covering a hydrophobic surface, said set of compositions comprising
a first composition consisting essentially of
stearylamine, especially a first composition of stearylamine at a concentration of from 0.1% to 0.5% or from 0.1% to 3%, in a suitable solvent, or
polyethyleneimine, especially a first composition of polyethyleneimine at a concentration of from 0.3% to 1% in a suitable solvent,
stearic acid, especially a first composition of stearic acid at a concentration of from 0.1% to 0.5% or from 0.1% to 3% in a suitable solvent, or
a second composition comprising or consisting essentially of hyaluronic acid, or a derivative thereof as defined above, at a concentration of from 0.1% to 0.5% or from 0.1% to 2% in water.

Said suitable solvent is especially water, heptane, dichloromethane, 2-methyltetrahydrofuran, dimethylformamide, dimethylacetamide or N,n′-dimethylpropyleneurea (DMPU), or alternatively a mixture thereof, in particular dichloromethane or 2-methyltetrahydrofuran as a mixture with dimethylformamide or dimethylacetamide or N,n′-dimethylpropyleneurea (DMPU).

In other words, the invention relates to the use of a set of compositions for covering a hydrophobic surface, said set of compositions comprising
a first composition consisting essentially of polyethyleneimine, especially polyethyleneimine at a concentration of from 0.3% to 1% in a suitable solvent, said suitable solvent especially being water or a polar solvent, dichloromethane, 2-methyltetrahydrofuran, dimethylformamide, dimethylacetamide or N,n′-dimethylpropyleneurea (DMPU), or alternatively a mixture thereof, in particular dichloromethane or 2-methyltetrahydrofuran as a mixture with dimethylformamide or dimethylacetamide or N,n′-dimethylpropyleneurea (DMPU), and
a second composition comprising or consisting essentially of hyaluronic acid at a concentration of from 0.1% to 0.5% or from 0.1% to 2% in water.

Also, the invention relates to the use of a set of compositions for covering a hydrophobic surface, said set of compositions comprising
a first composition consisting essentially of stearylamine, especially a first composition of stearylamine at a concentration of from 0.1% to 0.5%, or from 0.1% to 3% in a suitable solvent, or
a first composition consisting essentially of stearic acid, especially a first composition of stearic acid at a concentration of from 0.1% to 0.5%, or from 0.1% to 3% in a suitable solvent,

said suitable solvent especially being heptane, dichloromethane, 2-methyltetrahydrofuran, dimethylformamide or dimethylacetamide, or a mixture thereof, especially dimethylformamide as a mixture with dichloromethane, and
a second composition comprising or consisting essentially of hyaluronic acid, or a derivative thereof as defined above, especially oxidized hyaluronic acid or hyaluronic acid modified with adipic dihydrazide, or oxidized cellulose, or a mixture thereof, at a concentration of from 0.1% to 0.5% or from 0.1% to 2% in water.

Possibly, the set of compositions also comprises chromium III ions.

The invention also relates to the use of a set of compositions for covering a hydrophobic surface, said set of compositions comprising
a. a first composition consisting essentially of stearylamine or stearic acid, and
b. a second composition comprising or consisting essentially of hyaluronic acid, or a derivative thereof, especially oxidized hyaluronic acid or hyaluronic acid modified with adipic dihydrazide, or oxidized cellulose or a mixture thereof, at a concentration of from 0.1% to 2% in water.

The invention moreover relates to a kit for covering hydrophobic surfaces, comprising
a first composition consisting essentially of
   stearylamine, especially a first composition of stearylamine at a concentration of from 0.1% to 0.5% or from 0.1% to 3%, in a suitable solvent, or
   polyethyleneimine, especially a first composition of polyethyleneimine at a concentration of from 0.3% to 1%, in a suitable solvent,

said suitable solvent especially being water, heptane, dichloromethane, 2-methyltetrahydrofuran, dimethylformamide, dimethylacetamide or N,n′-dimethylpropyleneurea (DMPU), or alternatively a mixture thereof, in particular dichloromethane or 2-methyltetrahydrofuran as a mixture with dimethylformamide or dimethylacetamide or N,n′-dimethylpropyleneurea (DMPU), and
a second composition comprising or consisting essentially of hyaluronic acid at a concentration of from 0.1% to 0.5% or from 0.1% to 2% in water.

The invention also relates to a kit containing a container comprising:
a first composition consisting essentially of stearylamine or stearic acid, especially intended to be diluted in a suitable solvent to a concentration of from 0.1% to 3%,
a solvent or several solvents chosen from water, heptane, dichloromethane, 2-methyltetrahydrofuran, dimethylformamide, dimethylacetamide, N,n′-dimethylpropyleneurea (DMPU), 1,3-dioxolane, cyclopentyl methyl ether or a mixture thereof, and
a second composition comprising or consisting essentially of hyaluronic acid or a derivative thereof, especially oxidized hyaluronic acid or hyaluronic acid modified with adipic dihydrazide, or oxidized cellulose or a mixture thereof, at a concentration of from 0.1% to 2% in water.

The abovementioned kit thus comprises three components for covering hydrophobic surfaces. The kit moreover comprises an instruction notice on a suitable support (paper, information technology support, Internet link) for performing the steps of the process of the invention, as defined previously.

The invention will be understood more clearly on reading the examples that follow and the attached figures. These examples are given as examples and do not have any limiting nature on the invention.

### KEY TO THE FIGURES

FIG. 1 represents a Fourier transform infrared spectrum (FT-IR) of a polypropylene surface, as described in Example 1, which is untreated (curve 1), treated according to the process of the invention (curve 2) and hyaluronic acid alone (curve 3). The x-axis represents the wavelength in cm⁻¹ and the y-axis represents the transmission coefficient.

FIG. 2 represents a photograph showing a polypropylene plate treated on its left side with the process according to the invention (as described in Example 1; part 1. of the photo) and not treated on its right side (part 2. of the photo), and immersed in water. The white arrows indicate the separation zone between the treated part and the untreated part.

FIG. 3 represents a Fourier transform infrared spectrum of a polypropylene surface, as described in Example 2, which is untreated (curve 1), treated according to the process of the invention (curve 2) and hyaluronic acid alone (curve 3). The x-axis represents the wavelength in cm⁻¹ and the y-axis represents the transmission coefficient.

FIG. 4 represents a Fourier transform infrared spectrum of a polyurethane surface, as described in Example 3, which is untreated (curve 1), treated according to the process of the invention (curve 2) and hyaluronic acid alone (curve 3). The x-axis represents the wavelength in cm⁻¹ and the y-axis represents the transmission coefficient.

FIG. 5 represents a Fourier transform infrared spectrum of a polyurethane surface, as described in Example 4, which is untreated (curve 1), treated according to the process of the invention (curve 2) and hyaluronic acid alone (curve 3). The x-axis represents the wavelength in cm⁻¹ and the y-axis represents the transmission coefficient.

FIG. 6 represents a Fourier transform infrared spectrum of a Teflon® surface, as described in Example 5, which is untreated (curve 1), treated according to the process of the invention (curve 2) and hyaluronic acid alone (curve 3). The x-axis represents the wavelength in cm⁻¹ and the y-axis represents the transmission coefficient.

### EXAMPLES

### Example 1

### Treatment of a Polypropylene Plate with Stearylamine Followed by Coating with Hyaluronic Acid

A polypropylene plate is prewashed by immersing it vertically in a beaker containing 40 ml of dichloromethane and 40 ml of dimethylformamide. The beaker comprising the plate is ultrasonicated for two minutes and the beaker is then left shaking on an orbital shaker for one hour at room temperature (from 17.5° C. to 26° C.). The plate is removed from the washing mixture and left to dry in the open air for 15 minutes. Next, the plate is immersed vertically in a beaker containing 0.3% of stearylamine in 20 ml of dichloromethane and 20 ml of dimethylformamide. Under these conditions, the plate is treated over half its length and is placed in an oven thermostatically maintained at 26° C. on an orbital shaker for three hours. After this time, the plate is removed from the beaker and is immersed in three successive baths of distilled water for washing. Finally, the plate is immersed vertically in a beaker containing 80 ml of hyaluronic acid at 0.3% in distilled water. The beaker is shaken on an orbital shaker for two hours at room temperature. After this time period, the plate is removed and left to dry in the open air for 24 hours.

The surface of the plate is analyzed by FT-IR (FIG. 1). Only the part pretreated with stearylamine (curve 2) shows signals comparable to those of a hyaluronic acid gel taken as reference (curve 3).

The dried plate is again immersed in water for the purposes of a photograph (FIG. 2). Two zones are clearly distinct on this same plate: a zone not treated with stearylamine which is covered with a few droplets (part 2.) and a zone corresponding to the part treated with stearylamine, which is uniformly covered with a layer of hyaluronic acid gel (part 1.).

These experiments demonstrate that the plate treated with the process according to the invention shows good wetting, whereas the untreated plate remains relatively hydrophobic.

Moreover, the presence of hyaluronic acid covering the substrate is measured and assayed via a colorimetric test according to a protocol adapted from that described by Cesaaretti M. et al. 2003, Carbohydrate Polymers, 54, 59-61.

The protocol used is as follows: a given amount of the sample is placed in contact and covered with 2M sulfuric acid (400 microliters), ultrasonicated for one minute and then shaken on an orbital shaker for one hour. The sample is removed from the solution.

400 microliters of 25 mM sodium tetraborate in concentrated sulfuric acid are added to the remaining solution. The solution is heated at 100° C. for ten minutes and then allowed to cool to room temperature over 15 minutes. 200 microliters of a carbazole solution (0.125% in absolute ethanol) are then added. The solution is heated again at 100° C. for ten minutes and then allowed to cool to room temperature.

The characteristic coloration is evidence of the presence of hyaluronic acid (HA). The amount of hyaluronic acid is assayed on a spectrophotometer at a wavelength of 550 nm. When the surface has been treated, the solution is a translucent blue color, whereas it is colorless if the surface has not received any treatment.

The results obtained are represented in table 1 below:

TABLE 1

| Sample | OD |
|---|---|
| Solution without hyaluronic acid (HA) | 0.2359 |
| Solution containing 150 μg of HA | 0.5824 |
| Untreated polypropylene | 0.2257 |
| Polypropylene treated with stearylamine and coated with HA | 0.3362 |

This other test confirms the presence of HA on the treated surface.

### Example 2

### Treatment of a Polypropylene Plate with Polyethyleneimine Followed by Coating with Hyaluronic Acid

A polypropylene plate is prewashed by immersing it vertically in a beaker containing 80 ml of ethanol. The beaker comprising the plate is ultrasonicated for two minutes and the beaker is then left shaking on an orbital shaker for one hour at room temperature. The plate is removed from the washing mixture and left to dry in the open air for 15 minutes. Next, the plate is immersed vertically in a beaker containing 0.5% of polyethyleneimine in 40 ml of distilled water at room temperature. Under these conditions, the plate is treated over half its length and it is shaken on an orbital shaker for three hours. After this time, the plate is removed from the beaker and is immersed in three successive baths of distilled water for washing. Finally, the plate is immersed vertically in a beaker containing 80 ml of 0.3% hyaluronic acid in distilled water. The beaker is shaken on an orbital shaker for two hours at room temperature. After this time period, the plate is removed and left to dry in the open air for 24 hours. The surface of the plate is analyzed by FT-IR (FIG. 3). Only the part pretreated with polyethyleneimine (curve 2) shows signals comparable to those of a hyaluronic acid gel taken as reference (curve 3).

These experiments demonstrate that the plate treated with the process according to the invention shows good wetting, whereas the untreated plate remains relatively hydrophobic.

### Example 3

### Treatment of a Polyurethane Tube with Stearylamine Followed by Coating with Hyaluronic Acid

A polyurethane tube is prewashed by immersing it vertically in a beaker containing 80 ml of heptane. The beaker comprising the tube is ultrasonicated for two minutes and the beaker is then left shaking on an orbital shaker for one hour at room temperature. The tube is removed from the washing solvent and left to dry in the open air for 15 minutes. Next, the tube is immersed vertically in a beaker containing 0.3% of stearylamine in 40 ml of heptane. Under these conditions, the tube is treated over half its length and it is placed in an oven thermostatically regulated at 26° C. on an orbital shaker for three hours. After this time, the tube is removed from the beaker and it is immersed in three successive baths of distilled water for washing. Finally, the tube is immersed vertically in a beaker containing 80 ml of 0.3% hyaluronic acid in distilled water. The beaker is shaken on an orbital shaker for two hours at room temperature. After this time period, the tube is removed and left to dry in the open air for 24 hours.

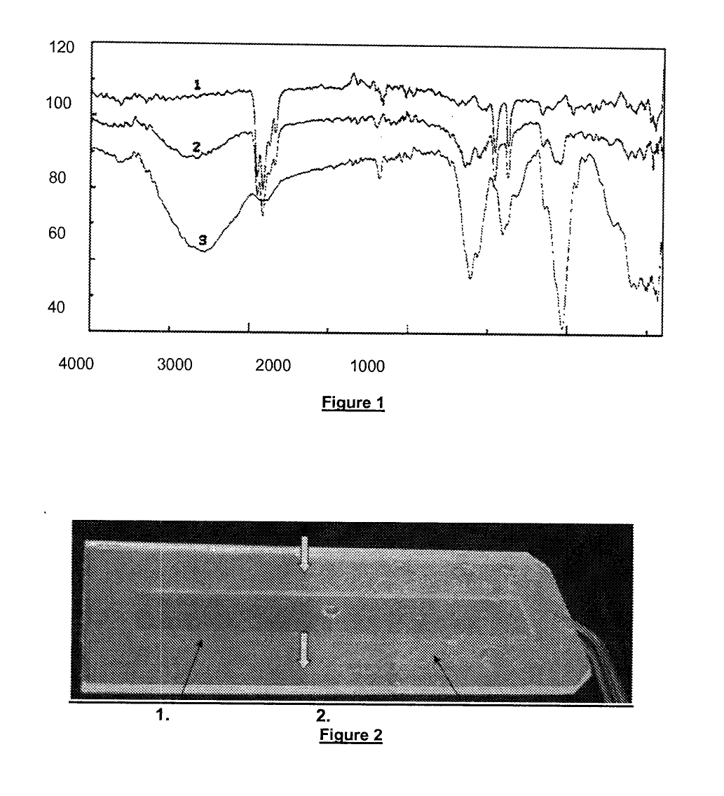

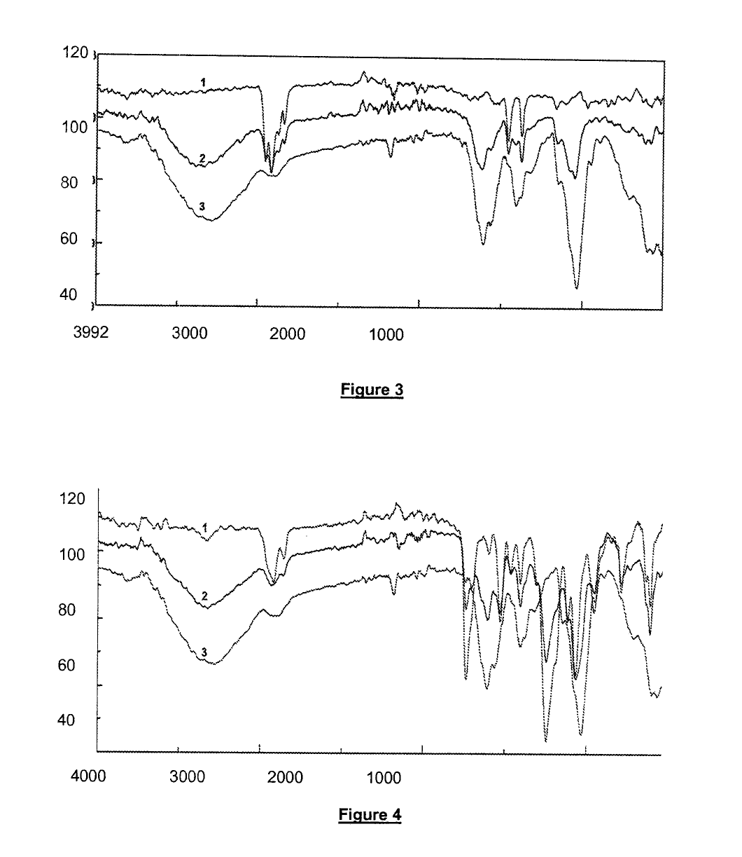

The surface of the tube is analyzed by FT-IR (FIG. 4). Only the part pretreated with stearylamine (curve 2) shows signals comparable to those of a hyaluronic acid gel taken as reference (curve 3).

The dried tube is again immersed in water for the wettability test. Two zones are clearly distinct: a zone not treated with stearylamine which is covered with a few droplets and a zone corresponding to the part treated with stearylamine, which is uniformly covered with a layer of hyaluronic acid gel.

These experiments demonstrate that the tube treated with the process according to the invention shows good wetting, whereas the untreated tube remains relatively hydrophobic.

### Example 4

### Treatment of a Polyurethane Tube with Polyethyleneimine Followed by Coating with Hyaluronic Acid

A polyurethane tube is prewashed by immersing it vertically in a beaker containing 80 ml of ethanol. The beaker comprising the tube is ultrasonicated for two minutes and the beaker is then left shaking on an orbital shaker for one hour at room temperature. The tube is removed from the washing solvent and left to dry in the open air for 15 minutes. Next, the tube is immersed vertically in a beaker containing 0.5% of polyethyleneimine in 40 ml of water at room temperature. Under these conditions, the tube is treated over half its length and it is shaken on an orbital shaker for three hours. After this time, the tube is removed from the beaker and it is immersed in three successive baths of distilled water for washing. Finally, the tube is immersed vertically in a beaker containing 80 ml of hyaluronic acid at 0.3% in distilled water. The beaker is shaken on an orbital shaker for two hours at room temperature. After this time period, the tube is removed and left to dry in the open air for 24 hours.

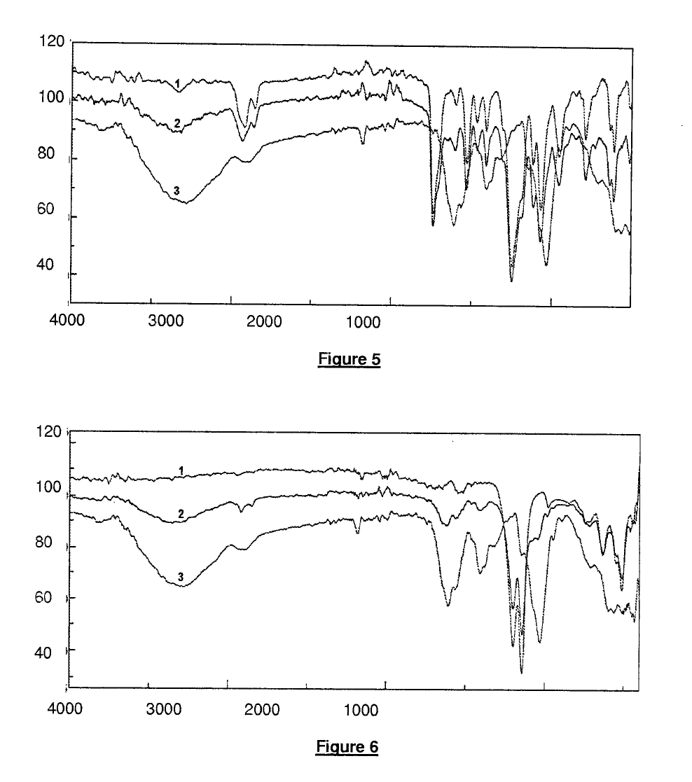

The surface of the tube is analyzed by FT-IR (FIG. 5). Only the part pretreated with polyethyleneimine (curve 2) shows signals comparable to those of a hyaluronic acid gel taken as reference (curve 3).

The dried tube is again immersed in water for the wettability test. Two zones are clearly distinct on this same plate: a zone not treated with polyethyleneimine, which is covered with a few droplets, and a zone corresponding to the part treated with polyethyleneimine, which is uniformly covered with a layer of hyaluronic acid gel.

These experiments demonstrate that the tube treated with the process according to the invention shows good wetting, whereas the untreated tube remains relatively hydrophobic.

### Example 5

### Treatment of a Teflon® Tube with Stearylamine Followed by Coating with Hyaluronic Acid

A Teflon® tube is prewashed by immersing it vertically in a beaker containing 40 ml of dichloromethane and 40 ml of dimethylformamide. The beaker comprising the tube is ultrasonicated for two minutes and the beaker is then left shaking on an orbital shaker for one hour at room temperature. The tube is removed from the washing mixture and left to dry in the open air for 15 minutes. Next, the tube is immersed vertically in a beaker containing 0.3% of stearylamine in 20 ml of dichloromethane and 20 ml of dimethylformamide. Under these conditions, the tube is treated over half its length and it is placed in an oven thermostatically regulated at 26° C. on an orbital shaker for three hours. After this time, the tube is removed from the beaker and it is immersed in three successive baths of distilled water for washing. Finally, the tube is immersed vertically in a beaker containing 80 ml of hyaluronic acid at 0.3% in distilled water. The beaker is shaken on an orbital shaker for two hours at room temperature. After this time period, the tube is removed and left to dry in the open air for 24 hours.

The surface of the tube is analyzed by FT-IR (FIG. 6). Only the part pretreated with stearylamine (curve 2) shows signals comparable to those of a hyaluronic acid gel taken as reference (curve 3).

The dried tube is again immersed in water for the wettability test. Two zones are clearly distinct on this same plate: a zone not treated with stearylamine, which is covered with a few droplets, and a zone corresponding to the part treated with stearylamine, which is uniformly covered with a layer of hyaluronic acid gel. These experiments demonstrate that the tube treated with the process according to the invention shows good wetting, whereas the untreated tube remains relatively hydrophobic.

### Example 6

### Treatment of a Teflon® Tube with Polyethyleneimine Followed by Coating with Hyaluronic Acid

A Teflon® tube is prewashed by immersing it vertically in a beaker containing 80 ml of ethanol. The beaker comprising the tube is ultrasonicated for two minutes and the beaker is then left shaking on an orbital shaker for one hour at room temperature (from 17.5° C. to 26° C.). The tube is removed from the washing solvent and is left to dry in the open air for 15 minutes. Next, the tube is immersed vertically in a beaker containing 0.5% of polyethyleneimine in 40 ml of water at room temperature. Under these conditions, the tube is treated over half its length and it is shaken on an orbital shaker for three hours. After this time, the tube is removed from the beaker and is immersed in three successive baths of distilled water for washing. Finally, the tube is immersed vertically in a beaker containing 80 ml of hyaluronic acid at 0.3% in distilled water. The beaker is shaken on an orbital shaker for two hours at room temperature. After this time period, the tube is removed and left to dry in the open air for 24 hours.

The dried tube is again immersed in water for the wettability test. Two zones are clearly distinct on this same plate: a zone not treated with polyethyleneimine, which is covered with a few droplets, and a zone corresponding to the part treated with polyethyleneimine, which is uniformly covered with a layer of hyaluronic acid gel. The colorimetric test described in Example 1 gives the results featured in table 2 below:

TABLE 2

| Sample | OD |
|---|---|
| Solution without HA | 0.2359 |
| Solution containing 150 μg of HA | 0.5824 |
| Untreated Teflon ® | 0.2273 |
| Teflon ® treated with polyethyleneimine and coated with HA | 0.3473 |

This test shows that the Teflon® tube covered with polyethyleneimine is covered with a layer of hyaluronic acid gel, whereas the untreated surface is not.

These experiments demonstrate that the tube treated with the process according to the invention shows good wetting, whereas the untreated tube remains relatively hydrophobic.

### Example 7

### Treatment of a Silicone Tube with Stearylamine Followed by Coating with Hyaluronic Acid

A silicone tube is prewashed by immersing it vertically in a beaker containing 40 ml of dichloromethane and 40 ml of dimethylformamide. The beaker comprising the tube is ultrasonicated for two minutes and the beaker is then left shaking on an orbital shaker for one hour at room temperature (from 17.5° C. to 26° C.). The tube is removed from the washing mixture and is left to dry in the open air for 15 minutes. Next, the tube is immersed vertically in a beaker containing 0.3% of stearylamine in 20 ml of dichloromethane and 20 ml of dimethylformamide. Under these conditions, the tube is treated over half its length and it is placed in an oven thermostatically regulated at 26° C. on an orbital shaker for three hours. After this time, the tube is removed from the beaker and is immersed in three successive baths of distilled water for washing. Finally, the tube is immersed vertically in a beaker containing 80 ml of hyaluronic acid at 0.3% in distilled water. The beaker is shaken on an orbital shaker for two hours at room temperature. After this time period, the tube is removed and left to dry in the open air for 24 hours.

The dried tube is again immersed in water for the wettability test. Two zones are clearly distinct on this same plate: a zone not treated with stearylamine, which is covered with a few droplets, and a zone corresponding to the part treated with stearylamine, which is uniformly covered with a layer of hyaluronic acid gel.

The colorimetric test described in Example 1 gives the results indicated in table 3 below:

TABLE 3

| Sample | OD |
|---|---|
| Solution without HA | 0.2359 |
| Solution containing 150 μg of HA | 0.5824 |
| Untreated silicone | 0.2656 |
| Silicone treated with stearylamine and coated with HA | 0.5416 |

This test shows that the silicone tube covered with stearylamine is covered with a layer of hyaluronic acid gel, whereas the untreated surface is not.

These experiments demonstrate that the tube treated with the process according to the invention shows good wetting, whereas the untreated tube remains relatively hydrophobic.

### Example 8

### Treatment of a Silicone Tube with Polyethyleneimine Followed by Coating with Hyaluronic Acid

A silicone tube is prewashed by immersing it vertically in a beaker containing 80 ml of ethanol. The beaker comprising the tube is ultrasonicated for two minutes and the beaker is then left shaking on an orbital shaker for one hour at room temperature (from 17.5° C. to 26° C.). The tube is removed from the washing solvent and is left to dry in the open air for 15 minutes. Next, the tube is immersed vertically in a beaker containing 0.5% of polyethyleneimine in 40 ml of water at room temperature. Under these conditions, the tube is treated over half its length and it is shaken on an orbital shaker for three hours. After this time, the tube is removed from the beaker and is immersed in three successive baths of distilled water for washing. Finally, the tube is immersed vertically in a beaker containing 80 ml of hyaluronic acid at 0.3% in distilled water. The beaker is shaken on an orbital shaker for two hours at room temperature. After this time period, the tube is removed and left to dry in the open air for 24 hours.

The dried tube is again immersed in water for the wettability test. Two zones are clearly distinct on this same plate: a zone not treated with polyethyleneimine, which is covered with a few droplets, and a zone corresponding to the part treated with polyethyleneimine, which is uniformly covered with a layer of hyaluronic acid gel. The colorimetric test described in Example 1 gives the results represented in table 4 below.

TABLE 4

| Sample | OD* |
|---|---|
| Solution without HA | 0.2359 |
| Solution containing 150 μg of HA | 0.5824 |
| Untreated silicone | 0.2656 |
| Silicone treated with polyethyleneimine and coated with HA | 0.3682 |

This test shows that the silicone tube covered with polyethyleneimine is covered with a layer of hyaluronic acid gel, whereas the untreated surface is not.

These experiments demonstrate that the tube treated with the process according to the invention shows good wetting, whereas the untreated tube remains relatively hydrophobic.

### Example 9

### Treatment of a Stainless-Steel Bar with Stearylamine Followed by Coating with Hyaluronic Acid

A stainless-steel bar is prewashed by immersing it vertically in a beaker containing 40 ml of dichloromethane and 40 ml of dimethylformamide. The beaker comprising the bar is ultrasonicated for two minutes and the beaker is then left shaking on an orbital shaker for one hour at room temperature (from 17.5° C. to 26° C.). The bar is removed from the washing mixture and left to dry in the open air for 15 minutes. Next, the bar is immersed vertically in a beaker containing 0.3% of stearylamine in 20 ml of dichloromethane and 20 ml of dimethylformamide. Under these conditions, the bar is treated over half its length and is placed in an oven thermostatically regulated at 26° C. on an orbital shaker for three hours. After this time, the bar is removed from the beaker and is immersed in three successive baths of distilled water for washing. Finally, the bar is immersed vertically in a beaker containing 80 ml of hyaluronic acid at 0.3% in distilled water. The beaker is shaken on an orbital shaker for two hours at room temperature. After this time period, the bar is removed and left to dry in the open air for 24 hours.

The dried bar is again immersed in water for the wettability test. Two zones are clearly distinct on this same plate: a zone not treated with stearylamine, which is covered with a few droplets, and a zone corresponding to the part treated with stearylamine, which is uniformly covered with a layer of hyaluronic acid gel.

The colorimetric test described in Example 1 also makes it possible to show that the stainless-steel bar covered with stearylamine is covered with a layer of hyaluronic acid gel, whereas the untreated bar is not.

The results obtained are indicated in table 5 below:

TABLE 5

| Sample | OD |
|---|---|
| Solution without HA | 0.2359 |
| Solution containing 150 μg of HA | 0.5824 |
| Untreated stainless steel | 0.2401 |
| Stainless steel treated with stearylamine and coated with HA | 0.5080 |

These experiments demonstrate that the bar treated with the process according to the invention shows good wetting, whereas the untreated bar remains relatively hydrophobic.

### Example 10

### Treatment of a Stainless-Steel Bar with Polyethyleneimine Followed by Coating with Hyaluronic Acid

A stainless-steel bar is prewashed by immersing it vertically in a beaker containing 80 ml of ethanol. The beaker comprising the bar is ultrasonicated for two minutes and the beaker is then left shaking on an orbital shaker for one hour at room temperature (from 17.5° C. to 26° C.). The bar is removed from the washing solvent and left to dry in the open air for 15 minutes. Next, the bar is immersed vertically in a beaker containing 0.5% of polyethyleneimine in 40 ml of water at room temperature. Under these conditions, the bar is treated over half its length and is shaken on an orbital shaker for three hours. After this time, the bar is removed from the beaker and is immersed in three successive baths of distilled water for washing. Finally, the bar is immersed vertically in a beaker containing 80 ml of hyaluronic acid at 0.3% in distilled water. The beaker is shaken on an orbital shaker for two hours at room temperature. After this time period, the bar is removed and left to dry in the open air for 24 hours.

The dried bar is again immersed in water for the wettability test. Two zones are clearly distinct on this same bar: a zone not treated with polyethyleneimine, which is covered with a few droplets, and a zone corresponding to the part treated with polyethyleneimine, which is uniformly covered with a layer of hyaluronic acid gel. The colorimetric test described in Example 1 gives the results represented in table 6 below:

TABLE 6

| Sample | OD |
|---|---|
| Solution without HA | 0.2359 |
| Solution containing 150 μg of HA | 0.5824 |
| Untreated stainless steel | 0.2401 |
| Stainless steel treated with polyethyleneimine and coated with | 0.5685 |
| HA | |

This test shows that the stainless-steel bar covered with polyethyleneimine is covered with a layer of hyaluronic acid gel, whereas the untreated bar is not. These experiments demonstrate that the tube treated with the process according to the invention shows good wetting, whereas the untreated tube remains relatively hydrophobic.

### Example 11

### Treatment of a COC (Cyclic Olefin Copolymer) Surface with Polyethyleneimine Followed by Coating with Hyaluronic Acid

A COC article is prewashed by immersing it vertically in a beaker containing 80 ml of ethanol. The beaker comprising the article is ultrasonicated for two minutes and the beaker is then left shaking on an orbital shaker for one hour at room temperature (from 17.5° C. to 26° C.). The article is removed from the washing solvent and left to dry in the open air for 15 minutes. Next, the article is immersed vertically in a beaker containing 0.5% of polyethyleneimine in 40 ml of water at room temperature. Under these conditions, the article is treated over half its length and it is shaken on an orbital shaker for three hours. After this time, the article is removed from the beaker and is immersed in three successive baths of distilled water for washing. Finally, the article is immersed vertically in a beaker containing 80 ml of hyaluronic acid at 0.3% in distilled water. The beaker is shaken on an orbital shaker for two hours at room temperature. After this time period, the article is removed and left to dry in the open air for 24 hours.

The dried article is again immersed in water for the wettability test. Two zones are clearly distinct on this article: a zone not treated with polyethyleneimine, which is covered with a few droplets, and a zone corresponding to the part treated with polyethyleneimine, which is uniformly covered with a layer of hyaluronic acid gel.

The colorimetric test described in Example 1 gives the results represented in table 7 below:

TABLE 7

| Sample | OD |
|---|---|
| Solution without HA | 0.2359 |
| Solution containing 150 μg of HA | 0.5824 |
| Untreated COC | 0.2538 |
| COC treated with polyethyleneimine and coated with HA | 0.3534 |

These experiments demonstrate that the COC article treated with the process according to the invention shows good wetting, whereas the untreated article remains relatively hydrophobic.

### Example 12

### Treatment of a Polyethylene Terephthalate (PET) Surface with Polyethyleneimine Followed by Coating with Hyaluronic Acid

A PET article is prewashed by immersing it vertically in a beaker containing 80 ml of ethanol. The beaker comprising the article is ultrasonicated for two minutes and the beaker is then left shaking on an orbital shaker for one hour at room temperature (from 17.5° C. to 26° C.). The article is removed from the washing solvent and left to dry in the open air for 15 minutes. Next, the article is immersed vertically in a beaker containing 0.5% of polyethyleneimine in 40 ml of water at room temperature. Under these conditions, the article is treated over half its length and is shaken on an orbital shaker for three hours. After this time, the article is removed from the beaker and is immersed in three successive baths of distilled water for washing. Finally, the article is immersed vertically in a beaker containing 80 ml of hyaluronic acid at 0.3% in distilled water. The beaker is shaken on an orbital shaker for two hours at room temperature. After this time period, the article is removed and left to dry in the open air for 24 hours.

The dried article is again immersed in water for the wettability test. Two zones are clearly distinct on this article: a zone not treated with polyethyleneimine, which is covered with a few droplets, and a zone corresponding to the part treated with polyethyleneimine, which is uniformly covered with a layer of hyaluronic acid gel. The colorimetric test described in Example 1 gives the results represented in table 8 below:

TABLE 8

| Sample | OD* |
|---|---|
| Solution without HA | 0.2359 |
| Solution containing 150 μg of HA | 0.5824 |
| Untreated PET | 0.2517 |
| PET treated with polyethyleneimine and coated with HA | 0.5137 |

This test shows that the PET surface covered with polyethyleneimine is covered with a layer of hyaluronic acid gel, whereas the untreated bar is not.

These experiments demonstrate that the article treated with the process according to the invention shows good wetting, whereas the untreated article remains relatively hydrophobic.

### Example 13

### Summary

Table 9 below summarizes the various tests performed in the context of the invention.

| | | | | | Coating |
|---|---|---|---|---|---|
| | | | | Rinsing | with |
| | | | | with | 0.3% |
| Ex. | Surface | Washing | Treatment | water | HA |
| 1 | Polypropylene | DCM/ | 0.3% ODA in | Yes | Yes |
| | | DMF (1/1) | DCM/DMF (1/1) | | |
| 2 | Polypropylene | Ethanol | 0.5% PEI in water | Yes | Yes |
| 3 | Polyurethane | Heptane | 0.3% ODA in | Yes | Yes |
| | | | heptane | | |
| 4 | Polyurethane | Ethanol | 0.5% PEI in water | Yes | Yes |
| 5 | Teflon ® | DCM/ | 0.3% ODA in | Yes | Yes |
| | | DMF (1/1) | DCM/DMF (1/1) | | |
| 6 | Teflon ® | Ethanol | 0.5% PEI in water | Yes | Yes |
| 7 | Silicone | DCM/ | 0.3% ODA in | Yes | Yes |
| | | DMF (1/1) | DCM/DMF (1/1) | | |
| 8 | Silicone | Ethanol | 0.5% PEI in water | Yes | Yes |
| 9 | Stainless steel | DCM/ | 0.3% ODA in | Yes | Yes |
| | | DMF (1/1) | DCM/DMF (1/1) | | |
| 10 | Stainless steel | Ethanol | 0.5% PEI in water | Yes | Yes |
| 11 | Cyclic Olefin | Ethanol | 0.5% PEI in water | Yes | Yes |
| | Copolymer | | | | |
| | (COC) | | | | |
| 12 | Polyethylene | Ethanol | 0.5% PEI in water | Yes | Yes |
| | terephthalate | | | | |
| | (PET) | | | | |

Key: DCM: dichloromethane, DMF: dimethylformamide, ODA: stearylamine (octadecylamine), PEI: Polyethyleneimine, HA: hyaluronic acid.

### Example 14

### Treatment of a Polypropylene Plate with Stearic Acid Followed by Coating with Hyaluronic Acid in the Presence of Chromium (III)

### a) Treatment

A polypropylene plate is prewashed by immersing it vertically in a beaker containing 80 ml of dimethylformamide. The beaker comprising the plate is ultrasonicated for two minutes and the beaker is then left shaking on an orbital shaker for one hour at room temperature (from 17.5° C. to 26° C.). The plate is removed from the washing mixture and left to dry in the open air for 15 minutes. Next, the plate is immersed vertically in a beaker containing 0.3% of stearic acid in 40 ml of dimethylformamide. Under these conditions, the plate is treated over half its length and is placed in an oven thermostatically regulated at 26° C. on an orbital shaker for three hours. After this time, the plate is removed from the beaker and is immersed in three successive baths of distilled water for washing.

### b) Coating

Next, the plate is immersed vertically in a beaker containing 80 ml of hyaluronic acid at 0.1% and 165 microliters of Cr(III) acetate hydroxide (basic chromium III acetate, CAS No. 3943-51-8) at 0.5% in distilled water. The beaker is left in an oven at 60° C. for two hours. Finally, the plate is immersed vertically in a beaker containing 80 ml of hyaluronic acid at 0.3% in distilled water. The beaker is shaken on an orbital shaker for one hour at room temperature. After this time period, the plate is removed and left to dry in the open air for 24 hours.

Two zones are clearly distinct on this article: a zone not treated with stearic acid, which is covered with a few droplets, and a zone corresponding to the part treated with stearic acid, which is uniformly covered with a layer of hyaluronic acid gel.

### Example 15

### Treatment of a Polypropylene Plate with Stearic Acid Followed by Coating with Hyaluronic Acid Substituted with Adipyl Dihydrazide (HA-ADH)

a) Preparation of the Hyaluronic Acid Substituted with Adipyl Dihydrazide (HA-ADH)

A solution of 200 mg of hyaluronic acid (Mw 1700 kDa) in 40 ml of 50 mM MES buffer (pH 4.6) is prepared. Next, 310 mg of 1-ethyl-3-(3-dimethylaminopropyl)carbodiimide (EDC) are added, followed by 2.61 g of adipic acid dihydrazide. The reaction is left to continue with magnetic stirring for two hours at room temperature. The reaction mixture is neutralized by adding 1N NaOH. Purification of the product is performed by dialysis. The dialysis (cellulose exclusion membrane Mw 3500) is started with 100 mM NaCl for 72 hours and then with 25% ethanol and finished with purified water. The product is obtained by freeze-drying. The method was described previously in: Prestwich et al. Journal of Controlled Release 1998, 53, 93-103.

### b) Treatment of a Polypropylene Plate

The treatment is that described in Example 14, in step a).

### c) Coating

Next, the plate is immersed vertically in a beaker containing 80 ml of hyaluronic acid coupled with adipyl dihydrazide at 0.5% in distilled water. The beaker is shaken on an orbital shaker for one hour at room temperature. After this time period, the plate is removed and left to dry in the open air for 24 hours.

Two zones are clearly distinct on this article: a zone not treated with stearic acid, which is covered with a few droplets, and a zone corresponding to the part treated with stearic acid, which is uniformly covered with a layer of hyaluronic acid gel coupled with adipyl dihydrazide.

### Example 16

### Treatment of a Polypropylene Plate with Stearic Acid Followed by Coating with Hyaluronic Acid Substituted with Adipyl Dihydrazide (HA-ADH) in the Presence of Oxidized Cellulose or Oxidized Hyaluronic Acid

### a) Preparation of Oxidized Cellulose and Oxidized Hyaluronic Acid

### Cellulose:

1 g of regenerated cellulose is taken up in 50 ml of deionized water. 1.32 g of sodium periodate are added. Next, the pH of the reaction mixture is adjusted to 3.5 by adding 1N HCl. The reaction is left to continue with magnetic stirring for five hours at 35° C.

### Hyaluronic Acid:

200 mg of hyaluronic acid are taken up in 20 ml of deionized water. 106 mg of sodium periodate are added. Next, the pH of the reaction mixture is adjusted to 3.5 by adding 1N HCl. The reaction is left to continue with magnetic stirring for 15 hours at room temperature. 300 μl of glycerol are added to the reaction mixture. Purification of the product is performed by dialysis in water for 72 hours. The product is obtained by freeze-drying.

The method was described previously in Kristiansen K. A. et al. Carbohydrate Research 2010, 345, 1264-1271

### b) Treatment of a Polypropylene Plate

The treatment is that described in Example 14, in step a).

### c) Coating

Next, the plate is immersed vertically in a beaker containing a mixture of 40 ml of HA-ADH at 0.5% and 40 ml of oxidized cellulose at 0.5% (or 40 ml of oxidized hyaluronic acid at 0.5%) in distilled water. The beaker is shaken on an orbital shaker for 15 minutes at room temperature. After this time period, the plate is removed and left to dry in the open air for 24 hours.

Two zones are clearly distinct on this article: a zone not treated with stearic acid, which is covered with a few droplets, and a zone corresponding to the part treated with stearic acid, which is uniformly covered with a layer of a mixture of hyaluronic acid gel coupled to adipyl dihydrazide and oxidized cellulose or oxidized hyaluronic acid.

1. A process for covering a hydrophobic surface with a hydrophilic composition, said process comprising:
placing in contact of the hydrophobic surface for the purpose of covering the hydrophobic surface with a first composition including
a solvent, and
a solute consisting of an amphiphilic compound of formula I below: 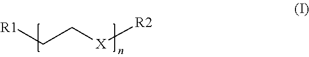
in which
n is equal to 1,
X is one of an amino group, an amido group, a carbonyl group, a carboxyl group, an ester group, a linear aromatic or polyaromatic group, a sulfonate group, a sulfate group, a phosphate group, and phosphonate group,
R2 is a hydrogen atom or a methyl, ethyl or propyl group, or is equal to R1,
and R1 is a saturated or unsaturated; C10-C24,
said amphiphilic compound including one of stearylamine, stearic acid, a salt thereof, and a derivative thereof,
said solvent being compatible with said amphiphilic compound and said hydrophobic surface,
rinsing with an aqueous solution of said hydrophobic surface, and
placing in contact of the rinsed surface with a second hydrophilic composition including a hydrophilic polymer.

2-3. (canceled)

4. The process according to claim 1, wherein said hydrophilic polymer is one of hyaluronic acid, alginate, oxidized cellulose, oxidized starch and derivative thereof.

5. The process according to claim 1, said hydrophilic polymer including hyaluronic acid at a concentration of from 0.1% to 2% in water.

6. The process for covering a hydrophobic surface with a hydrophilic composition according to claim 1, said process comprising
placing in contact of the hydrophobic surface with a first composition including
a solvent, and
a solute consisting of one of
stearylamine at a concentration of from 0.1% to 3%, and
stearic acid at a concentration of from 0.1% to 3%,
said solvent being compatible with said compound and said hydrophobic surface,
rinsing with water of said hydrophobic surface, and
placing in contact of the hydrophobic surface with a second hyaluronic acid composition at a concentration of from 0.1% to 2% in water.

7. A hydrophobic surface covered with hyaluronic acid obtained via the process of claim 6.

8. A hydrophobic surface covered with a first layer of an amphiphilic compound of formula I below: 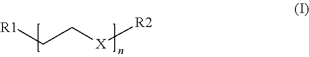
in which
n is equal to 1,
X is one of an amino group, an amido group, a carbonyl group, a carboxyl group, an ester group, a linear aromatic or polyaromatic group, a sulfonate group, a sulfate group, a phosphate group, and a phosphonate group,
R2 is a hydrogen atom or a methyl, ethyl or propyl group, or is equal to R1,
and R1 is a saturated or unsaturated C10-C24,
said amphiphilic compound including one of stearylamine, stearic acid, a salt thereof, and a derivative thereof, and
said first layer being covered with a second layer including a hydrophilic polymer.

9-10. (canceled)

11. The hydrophobic surface according to claim 8, wherein said hydrophilic polymer comprises hyaluronic acid, alginate, oxidized cellulose, oxidized starch, a derivative thereof or a mixture thereof.

12-13. (canceled)

14. A kit including a container comprising:
a first composition consisting essentially of stearylamine or stearic acid,
a solvent or a plurality of solvents including one of water, heptane, dichloromethane, 2-methyltetrahydrofuran, dimethylformamide, dimethylacetamide, N,n′-dimethylpropylenuerea (DMPU), 1,3-dioxolane or cyclopentyl methyl ether, and a mixture thereof, and
a second composition including hyaluronic acid or a derivative thereof, at a concentration of from 0.1% to 2% in water.

15. The process of claim 1, wherein R1 is one of C12-C20, C14-C18, and C16 alkyl.

16. The process of claim 6, wherein at least one of:
the stearylamine is at a concentration of 0.3%; and
the stearic acid is at a concentration of 0.3%.

17. The process of claim 8, wherein R1 is one of C12-C20, C14-C18, and C16 alkyl.

18. The kit according to claim 14, wherein the second composition includes one of an oxidized hyaluronic acid, a hyaluronic acid modified with adipic dihydrazide, an oxidized cellulose, and a mixture thereof.
